(19) United States
(12) Patent Application Publication
(10) Pub. No.: US 2019/0099345 A1
(43) Pub. Date: Apr. 4, 2019

(54) Use Of A Monosaccharide For Sub- And/Or Supragingival Tooth Cleaning

(71) Applicant: PRISMAN GmbH, Lorsch (DE)

(72) Inventors: Stephan Metzger, Lorsch (DE); Bjorn Schmalzbauer, Neu-Kupfer (DE); Kathrin Benzing, Heidelberg (DE)

(21) Appl. No.: 16/149,156

(22) Filed: Oct. 2, 2018

(30) Foreign Application Priority Data
Oct. 4, 2017 (EP) 17194745.0

(51) Int. Cl. A61K 8/60; A61K 8/02; A61Q 11/00; A61K 9/14; A61K 31/7004; A61K 9/00

(52) U.S. Cl. CPC A61K 8/60; A61K 8/022; A61Q 11/00; A61K 2800/28; A61K 31/7004; A61K 9/0063; A61K 2800/412; A61K 9/14

(57) ABSTRACT

The present invention refers to the use of at least one monosaccharide for the preparation of a powder for the sub- and/or supragingival powder jet cleaning of tooth surfaces. Furthermore, the invention refers to a monosaccharide for use in a dental jet powder for the therapy and/or prophylaxis of tooth-root or tooth diseases.

The present invention refers to the use of at least one monosaccharide for the preparation of a powder for the sub- and/or supragingival powder jet cleaning of tooth surfaces.

Within the framework of modern prophylaxis treatment, it is common today to clean the dental hard substance to remove hard and soft deposits. Powder jet cleaning is here a common procedure for the professional cleaning of tooth or tooth-root surfaces. Discolorations and plaque can thus be removed in a suitable way, whereby mechanical scraping with hand instruments or ultrasound treatments can be replaced or supplemented.

Tooth surface treatments are carried out using compressed air and fine powder particles, depending on the abrasive strength, in the form of a so-called air abrasion (conventional powder blasting) or air polishing (air-powder-water jet devices=LPW). Both processes are based on the principle that when a powder/compressed air mixture hits the tooth (root) surface, deposits adhering to it are removed. Airpolishing involves adding water to the air-powder mixture.

Sodium hydrogen carbonate has been used for over 20 years in powder jet cleaning as a component of the powders used for this purpose. Sodium hydrogen carbonate is well tolerated by the patients to be treated, but significant substance removal occurs when cleaning exposed root or dentin surfaces.

As an alternative to sodium hydrogen carbonate, sugar substitutes and crystalline amino acids were used as components of weakly abrasive powders in subgingival tooth surface cleaning.

Thus, EP 2228175 B1 discloses the use of a powder or powder mixture for the preparation of an agent for the powder jet cleaning of tooth surfaces, the powder containing an alditol, for example erythritol.

The use of fine-grained powders and/or powder mixtures for the preparation of an agent for the powder jet cleaning of supragingival tooth surfaces is further described in DE 10066408 B4. The powders or powder mixtures are here selected from amino acids, organic acids and their salts.

Furthermore, the use of a powder with a mean grain size of not more than 45 μm and a density of not more than 2.0 g/cm³ for the production of an agent for the powder jet cleaning of subgingival dental hard substance is disclosed in EP 1162940 B1.

Finally, DE 102014115412 A1 describes the use of a disaccharide as an abrasive and/or polishing body for the preparation of a dental jet powder for the supra- and/or subgingival powder jet cleaning of tooth surfaces.

However, many known powders have taste or physiological disadvantages and give patients an unpleasant mouthfeel or have a laxative effect.

It is therefore the object of the present invention to provide a powder for sub- and/or supragingival powder jet cleaning that has an optimized abrasive effect and improved patient acceptance.

This object is achieved by using at least one monosaccharide for preparing a powder for the sub- and/or supragingival powder jet cleaning of tooth surfaces.

In a preferred embodiment in combination with any of the above or below embodiments, the monosaccharide is selected from the group consisting of trioses, tetroses, pentoses and hexoses, in particular pentoses and hexoses.

In a preferred embodiment in combination with any of the above or below embodiments, the monosaccharide is selected from the group of trioses, in particular glyceraldehyde.

In a preferred embodiment in combination with any of the above or below embodiments, the monosaccharide is selected from the group of tetroses, in particular aldotetroses and ketotetroses.

In a preferred embodiment in combination with any of the above or below embodiments, the monosaccharide is selected from the group of aldotetroses, in particular erythrose and threose.

In a preferred embodiment in combination with any of the above or below embodiments, the monosaccharide is selected from the group of ketotetroses, in particular erythrulose.

In a preferred embodiment in combination with any of the above or below embodiments, the monosaccharide is selected from the group of pentoses, in particular aldopentoses and ketopentoses.

In a preferred embodiment in combination with any of the above or below embodiments, the monosaccharide is selected from the group consisting of aldopentoses, in particular ribose, arabinose, xylose and lyxose.

In a preferred embodiment in combination with any of the above or below embodiments, the monosaccharide is selected from the group consisting of ketopentoses, in particular ribulose and xylulose.

In a preferred embodiment in combination with any of the above or below embodiments, the monosaccharide is selected from the group of hexoses, in particular aldohexoses and ketohexoses.

In a preferred embodiment in combination with any of the above or below embodiments, the monosaccharide is selected from the group consisting of aldohexoses, more preferably allose, altrose, glucose, mannose, gulose, idose, galactose and talose, in particular galactose.

In a preferred embodiment in combination with any of the above or below embodiments, the monosaccharide is selected from the group consisting of ketohexoses, more preferably psicose, fructose, sorbose and tagatose, in particular tagatose.

In a preferred embodiment in combination with any of the above or below embodiments, the monosaccharide is selected from the group consisting of galactose and tagatose. These monosaccharides are particularly preferred because galactose and tagatose have a low glycemic index (galactose: 20, tagatose: 3). Furthermore, both sugars are considered “tooth-friendly”, wherein galactose is 20 times less cariogenic than glucose/fructose and tagatose is not cariogenic. The sugars, especially tagatose, also show a good spraying behavior.

All monosaccharides can be used in D or L form according to Fischer projection.

The monosaccharide acts as an abrasive and/or polishing body in the powder for the sub- and/or supragingival powder jet cleaning of tooth surfaces.

In a preferred embodiment in combination with any of the above or below embodiments, the powder has a mean grain size of <50 μm, more preferably 10-30 μm, especially 12-20 μm. These mean grain sizes have proven to be particularly advantageous in terms of conveying in the powder jet device and abrasive properties with respect to gums. A powder that is too fine can cause problems when being conveyed in the powder jet device and a powder that is too coarse is too abrasive or aggressive towards the gums.

According to the invention the monosaccharides are present as powders with a certain average particle size. The powder is produced in the desired grain distribution by grinding, sieving, air classifying, classifying and other process steps known to the skilled person. In addition to the particle velocity to be influenced by the corresponding device, factors such as the hardness and mass of the particles are substantially decisive for the abrasiveness of a jet powder. Large particles with a lower density have approximately the same abrasiveness with the same mass as smaller particles with a higher density. The grain geometry is also very important, with round grains being less abrasive than sharp-edged grains.

The mean particle size, as used herein, designates a mean volume-related particle size d(50) determined by the following method. The volume-related particle size was determined by laser diffraction with a particle size analyzer (Mastersizer 2000; light sources: red: helium-neon laser, 633 nm, blue: LED, 466 nm; particle size distribution spectrum: 0.02-2000 μm; software version 5.61, MALVERN Instruments Ltd, Malvern, UK). The cell selected was the Scirocco 2000 dry dispersion unit with a dispersion air pressure of 1 bar and a feed rate (vibrating chute) of 70% (MALVERN Instruments Ltd, Malvern, UK). The sample was taken and measured immediately after production of the monosaccharide. The evaluation of the raw data was based on the Fraunhofer theory. The volume-related particle size distribution is given here. The following mean particle sizes are the d(50) value, which is defined as the mean particle size. The d(50) value is understood to be the sum frequency distribution of the volume-averaged size distribution function. The d(50) value indicates that 50% of the measured particles have a diameter less than or equal to this value. This applies equally to the d(90) and d(10) value to be determined optionally. Here, 90% or 10% of the particles are smaller than or equal to the respective value. To characterize a particle size distribution, the value d(50) and optionally the values d(10) and d(90) are used. As explained above, the d(50) value describes the mean volume-related particle size, the values d(10) and d(90) describe the width of the volume-related particle size distribution.

In a preferred embodiment in combination with any of the above or below embodiments, the agent for the powder jet cleaning of sub- and/or supragingival dental hard substance contains at least one further substance selected from the group consisting of a flow aid, a bleaching agent, an analgesic, a bacteriocide, an alditol, a crystalline amino acid and/or a flavoring.

In a preferred embodiment in combination with any of the above or below embodiments, the flow aid is selected from the group consisting of silicon dioxide, calcium carbonate, aluminum silicate and/or aluminum hydroxide, more preferably silicon dioxide, in particular pyrogenic silicon dioxide (pyrogenic silica). Flow aids are separating agents which are added to crystalline substances in order to prevent the substances from clumping together and thus enable better machine use. The use of one or more flow aids may also be necessary for jet powders in order to avoid clogging of the nozzles of the jet device in the event of clumping of the powder substances.

In a preferred embodiment in combination with any of the above or below embodiments, the bleaching agent is selected from the group consisting of peroxide compounds, potassium, ammonium, sodium and lithium persulfates and perborate mono- and tetrahydrates, sodium pyrophosphate peroxyhydrate and magnesium, calcium, strontium and zinc peroxides.

In a preferred embodiment in combination with any of the above or below embodiments, the analgesic is selected from articaine or lidocaine.

In a preferred embodiment in combination with any of the above or below embodiments, the bacteriocide is selected from the group consisting of chlorhexidine, triclosan, copper, zinc and tin(ll) salts, such as zinc citrate, zinc sulfate, zinc glycinate, sodium zinc citrate and tin(ll)pyrophosphate, metronidazole, quaternary ammonium compounds, bisguanides such as chlorhexidine digluconate, hexetidine, cetylpyridinium chloride, octenidine and alexidine.

In a preferred embodiment in combination with any of the above or below embodiments, the alditol is selected from the group consisting of erythritol, sorbitol, xylitol, mannitol, isomalt, lactitol, threitol and arabitol. Alditols are non-cyclic polyols and can be used as sugar substitutes to improve taste. Alditols are not cariogenic.

In a preferred embodiment in combination with any of the above or below embodiments, the crystalline amino acid is glycine.

In a preferred embodiment in combination with any of the above or below embodiments, the flavoring is selected from the group consisting of natural or nature-identical odorants and/or flavors. These are generally understood to be flavorings which impart a particular smell or taste to products ingested or used orally. “Natural” flavoring odorants or flavors are substances or mixtures of substances obtained from natural sources, such as plants or parts of plants, and purified if necessary. Alternatively, the flavors to be used may also have been synthetically produced.

The other substances mentioned above must be selected with regard to their particle size in such a way that the mean grain size of the powder for the sub- and/or supragingival powder jet cleaning of tooth surfaces of <50 μm, 10-30 μm or 12-20 μm is retained.

The present invention also refers to a monosaccharide for use in a dental jet powder for the therapy and/or prophylaxis of tooth-root or tooth diseases.

In a preferred embodiment in combination with any of the above or below embodiments, the jet powder contains (i) 90-99.9% by weight of at least one monosaccharide and (ii) 0.1-10% by weight, more preferably (i) 95-99.5% by weight of at least one monosaccharide and (ii) 0.5-5% by weight, of at least one other substance selected from the group consisting of a flow aid, a bleaching agent, an analgesic, a bacteriocide and/or a flavoring.

For the monosaccharide to be used in a dental jet powder for the therapy and/or prophylaxis of tooth-root or tooth diseases, the same preferred embodiments as described for the use according to the invention are applicable.

Subgingival use, as used herein, is understood to mean the use of a jet powder on tooth surfaces below the gingival margin. A progressive inflammatory reaction caused by the accumulation of subgingival biofilms results in the formation of gum pockets that are virtually impossible to clean using a toothbrush or dental floss. Since subgingival tooth surfaces are softer and more sensitive than supragingival tooth surfaces, the abrasiveness of the jet powder used must be kept as low as possible.

Supragingival use, as used herein, means the use of a jet powder on tooth surfaces above the gingival margin.

If gums become inflamed and are not treated for a longer period of time, this can lead to inflammation of the periodontium, i.e. periodontitis. This is regularly accompanied by bone resorption and gum recession. As a result of the gum recession, the tooth necks are exposed and increased sensitivity occurs. These exposed tooth necks, as well as the usually subgingival areas, in particular the tooth roots, must be treated with powder of low abrasiveness in order not to injure the sensitive tooth tissue present here.

Regular cleaning of the tooth surfaces is essential for a healthy oral flora. In every mouth a coating forms on the teeth within a few days, called plaque or biofilm, which consists of billions of bacteria whose metabolic products can damage the teeth and gums. Similar to the development of caries, plaque on the tooth surface, in the interdental spaces and especially in the gum pockets is a major cause of inflammation of the gingival margin and the periodontium (periodontopathies).

An important goal of all tooth-conserving prophylactic measures to prevent tooth decay and periodontitis is therefore the prevention of the formation of plaque or the removal of plaque through regular and thorough dental care.

The use according to the invention as well as the monosaccharide according to the invention for use in a dental jet powder enable a successful therapy and/or prophylaxis of tooth-root or tooth diseases. The use according to the invention and the monosaccharide according to the invention for use in a dental jet powder achieve excellent cleaning and polishing performance with very low abrasiveness and excellent patient acceptance.

The invention is explained in the following by examples.

### EXAMPLE 1

100 g of the powder were prepared by mixing 99.05 g of a tagatose with a mean grain size d(50) of 15.97 μm (d(10)=2.39 μm, d(90)=49.04 μm), 0.45 g of pyrogenic silica and 0.50 g of peppermint flavor.

### EXAMPLE 2

100 g of a powder were prepared by mixing 99.05 g tagatose with a mean grain size d(50) of 10 μm, 0.45 g pyrogenic silica and 0.50 g peppermint flavor.

### EXAMPLE 3

100 g of a powder were prepared by mixing 98.80 g tagatose with a mean grain size d(50) of 20 μm, 0.20 g pyrogenic silica and 1.00 g cherry flavor.

### EXAMPLE 4

100 g of a powder were prepared by mixing 98.70 g tagatose with a mean grain size d(50) of 25 μm, 0.30 g pyrogenic silica and 1.00 g lemon flavor.

### EXAMPLE 5

100 g of a powder were prepared by mixing 98.00 g tagatose with a mean grain size d(50) of 30 μm, 1.00 g pyrogenic silica and 1.00 g lemon flavor.

### EXAMPLE 6

The tagatose powder according to Example 1 was compared with various known powders based on sodium hydrogen carbonate, glycine, erythritol and trehalose. Sodium hydrogen carbonate-based powders with a mean grain size of 40 μm are most commonly used in the cleaning of dental enamel. However, due to their high abrasiveness, sodium hydrogen carbonate powders are not suitable for cleaning sensitive tooth surfaces, tooth necks or in the subgingival area. The tagatose powder according to Example 1 with a mean grain size of 15 μm is less abrasive than conventional powders and has an excellent cleaning and polishing effect, as shown below.

The abrasiveness of the tagatose powder according to Example 1 was compared with commercially available powders (Air-Flow Classic Comfort, Air-Flow Soft, Air-Flow Plus, Air-Flow Perio from EMS Electro Medical Systems SA, Switzerland; Lunos Gentle Clean, Lunos Perio Combi from Dürr Dental AG/Orochemie GmbH+Co. KG, Germany) as shown in Table 1. The tests were carried out with a commercially available powder jet device (Airflow handy 2+, EMS Electro Medical Systems SA, Switzerland). The polyetheretherketone PEEK, a high-performance polymer used in dental prosthetics, was used to simulate the tooth surface. The nozzle of the powder jet device was locked at a distance of approx. 2 mm above the polymer surface and the powder jet was activated for 30 seconds at a jet pressure of 4.0 bar. The depth of the area of the polymer surface ground off by the powder used served as a measure of its abrasiveness. Each test was repeated five times to take account of statistical fluctuations. The value of 10 corresponds to the abrasiveness of sodium hydrogen carbonate with a mean grain size of 40 μm (see Table 1). A lower numerical value corresponds to a lower abrasiveness.

TABLE 1
Abrasiveness of various powders

| | Mean grain size | | | | |
|---|---|---|---|---|---|
| | Ø 65 μm | Ø 40 μm | Ø 30 μm | Ø 25 μm | Ø 16 μm |
| Sodium | | 10 | | | |
| hydrogen | | | | | |
| carbonate | | | | | |
| Glycine | 9 | | | 6 | |
| Trehalose | 4 | | 2 | | |
| Erythritol | | | | | 3 |
| Tagatose | | | | | 1 |

In addition, the cleaning and polishing effect of the tagatose powder used according to the invention according to Example 1 was tested in comparison to commercially available powders. For this purpose, the removal of dental plaque was simulated. The tests were carried out with a commercially available powder jet device (Airflow handy 2+, EMS Electro Medical Systems SA, Switzerland). The tagatose powder according to the invention and the powders to be compared were blasted onto a coated PEEK plate. Areas measuring 1 cm×1 cm were treated with the respective powders until the coating was completely removed from the surface. The time required to clean the surface was measured. In order to take statistical fluctuations into account, each test was repeated ten times. For better comparability of the cleaning effect and the polishing effect, the value 10 was assigned to the sodium hydrogen carbonate powder with a particle diameter of 40 μm, as in the previous test. The times determined were normalized to the value 10. The higher the point value, the better the cleaning performance (see Table 2). A particularly gentle cleaning and polishing effect of the tagatose powder according to Example 1 used according to the invention was shown.

TABLE 2
Cleaning effect of various powders

| | Mean grain size | | | | |
|---|---|---|---|---|---|
| | Ø 65 μm | Ø 40 μm | Ø 30 μm | Ø 25 μm | Ø 16 μm |
| Sodium | | 10 | | | |
| hydrogen | | | | | |
| carbonate | | | | | |
| Glycine | 13 | | | 15 | |
| Trehalose | 14 | | 13 | | |
| Erythritol | | | | | 14 |
| Tagatose | | | | | 17 |

To assess the polishing effect, the optics and haptics of the surface after blasting of the coated area were also evaluated. Here, too, the value 10 was assigned to the sodium hydrogen carbonate powder with a particle diameter of 40 μm for better comparability. The smaller the point value, the smoother and more uniform the cleaned surface (see Table 3).

TABLE 3
Polishing effect of various powders

| | Mean grain size | | | | |
|---|---|---|---|---|---|
| | Ø 65 μm | Ø 40 μm | Ø 30 μm | Ø 25 μm | Ø 16 μm |
| Sodium | | 10 | | | |
| hydrogen | | | | | |
| carbonate | | | | | |
| Glycine | 8 | | | 2 | |
| Trehalose | 9 | | 5 | | |
| Erythritol | | | | | 3 |
| Tagatose | | | | | 2 |

The tagatose powder according to Example 1 used according to the invention showed an excellent cleaning and polishing performance with very low abrasiveness. The glycine powder with a particle diameter of 25 μm as well as the erythritol powder with a particle diameter of 15 μm showed a similar behavior when polishing the surface, however, these did not perform as well when evaluating the cleaning time and the abrasiveness compared to the tagatose powder used according to the invention. This polishing effect makes a final polishing of the tooth after blasting superfluous.

Tagatose has a low glycemic index (GI) and is suitable for diabetics. Tagatose is also very tooth-friendly and not cariogenic. It shows a high tolerability and does not have a laxative effect. Tagatose is still not hygroscopic and shows an excellent spraying behavior, there is no caking of the powder in the jet device.

1. A method to clean tooth surfaces comprising sub- and/or supragingival powder jet cleaning of said tooth surfaces with a powder comprising at least one monosaccharide.

2. The method according to claim 1, wherein the monosaccharide is selected from the group consisting of pentoses and hexoses.

3. The method according to claim 1, wherein the monosaccharide is a ketohexose.

4. The method according to claim 1, wherein the monosaccharide is tagatose.

5. The method according to claim 1, wherein the monosaccharide is an aldohexose.

6. The method according to claim 1, wherein the monosaccharide is galactose.

7. The method according to claim 1, wherein the powder has a mean grain size of <50 μm.

8. The method according to claim 7, wherein the powder has a mean grain size of 10-30 μm.

9. The method according to claim 1, wherein the powder further comprises a flow aid, a bleaching agent, an analgesic, a bacteriocide, an alditol, a crystalline amino acid or a flavoring aid, or any combinations thereof.

10. Monosaccharide for use in a dental jet powder for the therapy and/or prophylaxis of tooth-root or tooth diseases.
